(19) United States
(12) Patent Application Publication
(10) Pub. No.: US 2010/0318109 A1
(43) Pub. Date: Dec. 16, 2010

(54) PROSTHESIS FOR ANASTOMOSIS

(76) Inventor: Luiz Gonzaga Granja Filho, Recife/PE (BR)

Correspondence Address: VOLPE AND KOENIG, P.C., UNITED PLAZA, 30 SOUTH 17TH STREET, PHILADELPHIA, PA, 19103, US

(21) Appl. No.: 12/303,833

(22) PCT Filed: Jun. 6, 2007
(86) PCT No.: PCT/BR2007/000144

§ 371 Date: Aug. 2, 2010

(30) Foreign Application Priority Data
Jun. 6, 2006 (BR) PI 0602382-7

(51) Int. Cl. A61B 17/11

(52) U.S. Cl. 606/153

(57) ABSTRACT

Prosthetic devices are provided that are used in end-to-side, end-to-end, and side-to-side anastomosis without clamping and sutureless, or with clamping and sutureless, where the graft is inserted in at least one of the intraluminal parts of the tubular member of the prosthesis, and is everted and coated, being previously fixed to the flange. The tubular member and flange are screwed among them in order to make the size of the intraluminal part more flexible.

### FIELD OF INVENTION

The present invention relates in a general manner to anastomotic devices and more specifically to a prosthetic device that allows anastomosis without clamping and sutureless, or with expeditious clamping and sutureless (in organs with normal walls, not friable or calcified), where a vascular graft, or any other tube, is inserted in the lumen of the prosthesis and reversed by jacketing to cover part of the prosthesis, said intraluminal. After the eversion, it will be definitely fixed to the flange, with points that pass through its holes or openings. The prosthesis flange has a plurality of spaced openings in its peripheral part, allowing the prosthesis to be sutured in the tissue, vein, artery or any other organ, out of the anastomosis, then, eliminating one of the main causes of stenosis and/or obstruction of the anastomosis which is the introduction of foreign bodies inside the lumen, due to the reaction to foreign bodies that occurs at the anastomosis site and also eliminating clamping, which is the main factor of thromboembolisms and tissue lacerations, specially of friable structures and/or calcified. Due to the fact that it is screwed, it allows a perfect match of the intraluminal part of the prosthesis with the wall thickness of the organ, safely avoiding any risk of protuberance in the prosthesis-graft aggregate under the light of the organ.

### DESCRIPTION OF THE PRIOR ART

A prior art presents several trials provide solutions for anastomotic devices projected to correct vascular abnormalities, which present the following typical features:

The North-American U.S. Pat. No. 3,254,650, of Jun. 7, 1966, describes a method and devices to execute anastomosis procedures by applying with adhesive two separated connectors in a body member and removing this body member portion contained among the connectors, joining the said connection devices for joining the remaining portions of the body member.

The U.S. Pat. No. 3,265,069, of Aug. 9, 1966, describes devices or instruments for use by surgeons in reunion of body ducts, which in the course of operations were separated. The instruments comprise a pair of elongated similar elements and articulatedly connected, in an intermediary manner, and with an support for finger retention in a distal end, comprising a generally cylindrical shape with a cylindrical channel that passes through it in the other distal end, in order to receive tubular body ducts kept by the instrument while the body ducts are reconnected.

U.S. Pat. No. 3,774,615, of Nov. 27, 1973, describes a device to connect the end of interrupted tubular organs without sewing, comprising a connecting ring on which the end of the interrupted organ are pulled, the ring is preferably locked up by a fixation resource. The ring and fixation resource are made of inert material, and preferably a hydrophile gel that can be dilated until its equilibrium or can be a hydrogel incompletely dilated, which is submitted to additional dilatation where it is applied. The connecting ring can be supplied with a groove and can be placed in a ring shaped fixation resource and kept there joining it to the fixation resource in the groove or simply kept by a screw. Two connection rings can also be used and kept joined by a coupling member.

The document U.S. Pat. No. 4,366,819, of Jan. 4, 1983, describes an anastomotic joint for surgery with a graft of coronary artery deviance comprising a mounting of four elements including a cylindrical tube with at least one locking indentation of ring flange in one influx end and a plurality of grooves of locking ring in a flow end; a ring flange with a central opening and a plurality of long and short spigots, the long spigots are engaged in the locking indentation, with a graft engaged among them; a fixation ring with a central opening and a plurality of spigots positioned around the opening; and a locking ring with a opening with a plurality of locking ring edges for engaging with the locking ring grooves. In surgical implants, an aortic wall with a hole engages between the ring flange and the fixation ring and is kept in this position by spigots of the fixation ring, and the four elements engage together forming an integral anastomotic joint. A first alternate modality includes an anastomotic joint of three elements with a combination of fixation ring and locking ring. A second alternate modality includes an anastomotic joint of four elements with a slightly jolted end in a influx end, exposing the graft material in the anastomotic “ostium”.

Other prior arts are equally mentioned, base don some information of “The Cardiothoracic Surgery Network”. The “Symmetry Aortic Connector System”, developed by St. Jude Medical, is a connector made with nitinol, selected by vein diameter with an adventitia removed to allow adjust of the connector and to prevent its displacement by the blood current. Then, the device may make an angle of 90° with the aorta. Among the disadvantages, there is the fact that it can be used only in extreme cases due to the difficult usage of this technique; it did not obtain a satisfactory result in many surgeries and it is being drowned out of market by the manufacturer; it is not applicable in calcified aorta; presents suture; presents contact with blood flow (foreign body); it does not widen the anastomosis area (restrictive anastomosis); performs only one anastomosis at a time; it is a product restrict to end-to-side anastomosis; a great mobilization of the venous graft occurs, damaging it, and can eventually form thrombus; there is a risk of perforation of the posterior wall of aorta; and the adventitia is removed (most resistant vascular layer).

Other known device is the PAS-Port™ System, a device used in 3 steps, and the vein wall is mounted over the device and is manually reversed on it, by tool and adapted to aorta with a angle of 90°. The method alerts that the surgeon shall select with due care the point of aorta and the vein size. The device is made of stainless steel and is available in only one size that allows the use of veins with external diameter of 4 to 6 mm, aorta with an internal diameter of 18 mm. It is available in only one size, limiting its applicability. As disadvantages of this prior art, the device has contact with blood flow (foreign body); it does not widen the anastomosis area (restrictive anastomosis); it uses veins with external diameter of 4 to 6 mm and aorta with an internal diameter of 18 mm; it does not perform multiple nor visceral anastomosis; it performs just only end-to-side anastomosis; a great mobilization of the used biological graft occurs, damaging its inner layer, which generates the formation of thrombus; there is a big risk of kinking at the origin (angle of 90°) and risk of posterior wall perforation in the aorta at the moment the device is introduced under its light; the suture is substituted with disadvantages by stainless steel (9 pins, distant among them, maximizing the risk of bleeding).

Also as prior art, there is the CorLink Device, currently commercialized by Ethicon/Johnson & Johnson, that allows the creation of anastomosis between the ascending aorta and a saphenous vein segment. Aortic Anastomotic Device (AAD) is a self-expanded device with extra luminal nitinol constituted by a de urn central cylinder with five interconnected elliptical arches and 2 groups of 5 pins in the end portion of the cylinder. The pins, after the eversion of venous walls in the device, fix the aggregate penetrating into the venous graft wall. A blade makes an opening in the wall of aorta and permits the coupling of AAD, which also fix the wall of aorta by pins. With this device: it poses a serious risk of bleeding, especially in friable aortas, thin, calcified or fibrous, restricting its applicability, also with risks, even in aortas with normal walls; in small gauge anastomosis, there is a risk of thrombosis, hyperplasia, intimal proliferation and fibrosis (reaction to foreign body type in origin of anastomosis) with consequent stenosis resulting in occlusion of anastomosis; sutures are used in some cases; there is cases of infarction caused by equipment; there is a recurring need of re-operations in patients; the device presents contact with blood flow (foreign body); it is not flexible; it does not multiple anastomosis; an inadequate mobilization of venous graft occurs, and can cause damage to its intimal layer, it could form thrombus; it is used only in extreme cases because it is a technique of complex usage; the suture is substituted by stainless steel in contact with blood flow.

Another known device is the St. Jude Distal Connector that consists of a stainless steel clip mounted on a catheter, comprising a balloon for subsequent expansion and connector mounting. The catheter is introduced backward from the end, by doing a small hole in the anastomosis site, the clip fixes the vein in the hole, the catheter goes to coronary and releases the connector. The catheter is removed and a suture is done in side-to-side anastomosis. With St. Jude Distal Connector, occurrence of leakage problems were detected in 20% of the used connectors; the use of a metallic clip requires due care for handling to avoid distortion in the anastomosis; late angiographies reveal smaller circular diameter of anastomosis made with o St. Jude Distal Connector, when compared to controls made with conventional suture; there is remarkably risk of bleeding and the graft is very mobilized, and lacerations can occur in its inner layer, allowing the formation of thrombus.

The HeartFlo™ is a multi-suture instrument for anastomosis with wires automatically applied in end-to-side and side-to-side anastomosis. The surgeon manually ties the suture wires (10 wires) and concludes the anastomosis similarly to the traditional process. Besides of being a product of complex handling, it makes suture in anastomosis (keeping the undesirable foreign body in the internal origin of the anastomosis) and is restricted to end-to-side and side-to-side anastomosis. There is also an excessive mobilization of graft, and can cause lesions in its intimal layer, which would be the inductor that forms the thrombus.

Another technique and known device is the Solem Graft connector, produced by the Swedish company Jomed. It is constituted by a stent made of nickel and titanium coated with polytetrafluorethylen used to connect the internal thoracic artery the left anterior descending coronary artery. The results has not been satisfactory, because it poses risk of bleeding; there is also an excessive mobilization of graft, probably damaging intimal layers, allowing the formation of thrombus; it is not flexible, by this fact, causes trauma to grafts; it does not make multiple anastomosis, at a single time; presents contact with blood flow (foreign body); and is frequent the need of-operations.

The Magnetic Vascular Positioner System is produced by Ventrica and comprises 4 magnetic rings and the anastomosis is processed by magnetic attraction of 4 ports. However, initial experimental results demonstrate leakage, also a undesired contact of materials with blood flow. On the other hand, it is necessary to be careful to avoid the capitation of excess of tissue among the magnets. With this system, there is also a need of suture in some cases; there is occurrence of infarction caused by equipment; and is frequent the need of-operations in patients; and also requires clamping.

Also, as a device known by the medical area, the Combined Anastomotic Device and Tissue Adhesive, developed by Grundeman & Borst group, combines micro mechanical technique with use of adhesive (glue). The use of this method can result in leakages and need traditional sutures; it is frequent the need of reoperation due to leakage/bleeding; and performs only one anastomosis at a time.

Finally, it is also experimentally practiced anastomosis assisted by laser, where the results are not different from conventional isolated sutures, because there is a need of suture in some cases; there is a risk of bleeding e leakage; and does not perform multiple anastomosis.

Even so divulged nowadays, anastomosis with damper, by insecurity, and almost totality of surgeons perform conventional sutures throughout the route of anastomosis, with an intention of avoiding leakages and bleedings, it means the use of dampers just makes the procedure more expensive, once the conventional suture is also applied.

In short, the conventional anastomosis, with clamping and with suture, standardized in 1906 by Aléxis Carrel, remains the first choice for any type of anastomosis and organs to be anastomosed.

With an expectation of changing the current situation, the Brazilian patent no. PI 9706197-2, describes and claims a prosthesis for vascular anastomosis, or in any other organ or tissue, without the use of clamping and sutureless, solving, in an elegant and efficient manner, the limitations inherent to prosthesis of the above mentioned prior art, when used in vascular anastomosis performed, mainly in thin aortas, calcified and friable; or in any other application where a clamping of a vein or artery can pose excessive trauma for conditions of a given patient. The prosthesis that is subject of that request allows the embodiment of fast and safe anastomosis, without obstruction of vein or artery lumen of which anastomosis is made, also allows anastomosis in tissues, veins or arteries in bad conditions and never would accept a clamping used in conventional anastomosis. This is achieved by a generally cylindrical shaped prosthesis with a flange orthogonally extending from its external side wall, in a point in the prosthesis length between its ends; the referred flange has openings distributed around its surface. The description of the usage method and specific construction of the prosthesis is presented in the drawings of the descriptive report of that request, as well as the document C19706197-2, Certificate of Addition of the first.

Although these anastomotic devices can be presented as suited to the purposes for which they were projected, they are not so suited for the purposes of the present invention, as described herein below.

### SUMMARY OF THE INVENTION

The present invention relates to variations of the current known anastomotic devices, in order to allow side-to-side, end-to-end e end-to-side anastomosis without clamping e sutureless, or with expeditious clamping and sutureless in organs of normal walls, where at least one vascular graft, or any other, is inserted in the lumen of the prosthesis and reversed by jacketing to cover part of the prosthesis, said intraluminal, being however fixed to the flange by points that pass through holes or openings. The tubular member of the prosthesis also has an external screw to be screwed to the flange, making the size of the intraluminal part more flexible. The flange has openings, allowing the fixation of the everted part of the graft and to fixate it to the exterior of the tissue, vein, artery or tubular organ to avoid the contact of foreign bodies with the interior of the anastomosis. The prosthesis can also present varied sizes and shapes to simultaneously accommodate varied numbers, sizes and types of grafts.

One objective of the present invention is to provide an anastomotic device with a screwed tubular member and a screwed flange, allowing the insertion of more than one graft, of different types and gauges (for example, anastomotic trunk formed by junction of one of its ends, of multiple biologic grafts such as autolologs, homologs, heterologs or synthetics) in the said prosthesis.

Another objective of the present invention is to provide an anastomotic device that does not introduces any foreign body inside the anastomosed grafts.

A further objective of the present invention is to provide an anastomotic device which fixation of the everted graft segment is made by points in the flange, externally placed and not in the intraluminal, tubular member.

Also, it is an objective of the invention to allow the variation of size of the prosthesis intraluminal part according to the wall thickness of the organ that receives the anastomotic aggregate, eliminating the risk of protuberance of prosthesis-graft aggregate under the light of the organ.

Other features and additional objectives of the present invention will become apparent from the following descriptions. These features will be described on sufficiently detailed levels to allow the technicians of the subject matter to implement the invention. Also, it is understood that other features can be used and structural changes can be made without leaving the scope of the invention. In the accompanying drawings, like reference numbers indicate identical or like parts throughout the several views.

Therefore, the following detailed description should not be taken as limiting the scope of the present invention which is defined by the appended claims.

### BRIEF DESCRIPTION OF THE DRAWINGS

The present invention may be understood more completely by reference to the following description and appended drawings, supported by examples, in which:

FIG. 1 illustrates a first embodiment of the prosthesis for anastomosis with flanges and screwed intraluminal parts, comprising removable or fixed rods to facilitate screwing.

FIG. 2 illustrates a second embodiment of the prosthesis for anastomosis with flanges and screwed intraluminal parts, also comprising a screwed prosthesis mandrill that is removed after the anastomosis is concluded.

FIG. 3 illustrates another structural embodiment of the screwed prosthesis with chamfers in the flange, substituting holes.

### DESCRIPTION OF THE PREFERRED EMBODIMENTS

With reference to the drawings, in which like reference numbers indicate identical elements throughout the several views, the figures illustrate one form of the present invention, in the form of a prosthesis for anastomosis with flange and screwed tubular body.

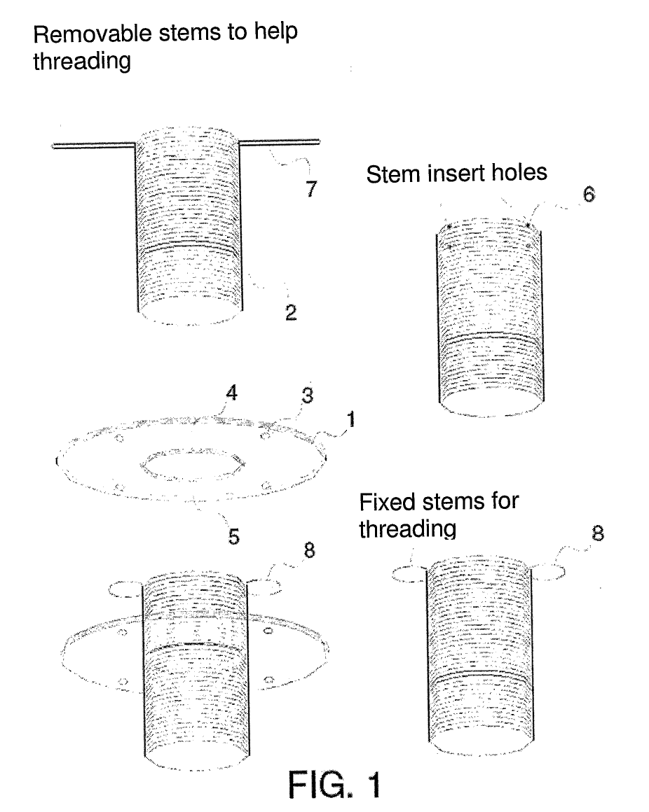

FIG. 1 presents a prosthesis comprising two parts, a flange 1 and a tubular member 2. The flange 1 has at least four passing through holes or openings 3 and a central edge 4 comprising a internal screw 5. The tubular member 2 has an external screw to be adapted, by screwing, to the flange 1. It has also obliquous or perpendicular holes 6 in the upper edge, for joining the removable rods 7 to help screwing. The rods 8 can also be fixed to the tubular member 2, avoiding the inadverted disconnection of the flange 1 by the upper end. Therefore, it would have a slight groove on the upper surface of the flange to accommodate the rods, if it is to be screwed until the end. The utilization technique consists of, initially, passing the graft or anastomotic trunk through the light of the flange and fixing the edges with points. Then, the distal end of the grafts is passed backward through the light of the tubular member, until it is juxtaposed to the flange. The tubular member 2 is slightly screwed to the flange 1 until its maximum limit. The whole tubular member 2 is coated with graft or anastomotic trunk that is automatically everted while proceeding the screwing. Passing through the points of the flange 1 and it is applied in predetermined places on the surface of the organ wall. There is an incision of the wall through the points, a temporary tamponade is made, and the anastomotic aggregate is introduced and fixed, by tying the points to the wall of the organ. Next, also slightly, the tubular member 2 is descrewed until the desired height, aligned to the internal surface level of the organ wall, without any protuberance. By digital palpation, the tubular member 2 is easily turned to one side, measuring the anastomotic aggregate depth, introducing or removing it, as needed. If it is inadvertidely out of the flange 1, just screw it again slightly. Other technical embodiment can comprise the use of local ecodoppler or transesofagic to determine this depth, which is certainly not necessary. This is because the screwing limit can be determined, setting the thickness of the organ wall at the anastomosis point, by using in a simple and cheap manner, a needle with a point bent in 90 grades. The needle is introduced, tractioned until reaching the internal surface of the wall, externally fixing that point, then removing it and the depth is measured. So, the tubular member 2 would be screwed until that extension. We mention again that all of this is dispensable just by performing the palpation in the origin of the anastomosis by turning the tubular member to one side.

The flange of the prosthesis in FIG. 1 has, besides the holes, double holes or handles 10 on the upper surface that are useful to anchor the suture wire legs, in order to avoid the interposition in the prosthesis light, even in prosthesis with big gauge. Also, the flange can have a minimum extension, just enough to accommodate the internal, external holes, and double holes. When the organ wall is very friable, calcified or fibrous, the preparation of the double suture of the flange 1 to this wall can avoid the laceration when the wires are tied-up. The wire comes from internal holes, with points in U, coming in and out through the double hole that anchor the wire in its middle, transfixing or not the wall and returns, from bottom to top to the flange by the external holes. Then, they are tied in the upper surface of the flange, keeping the wall almost in its original position, assuring its integrity. Also, this double suture reinforces hemostasis. The four points are equally applied.

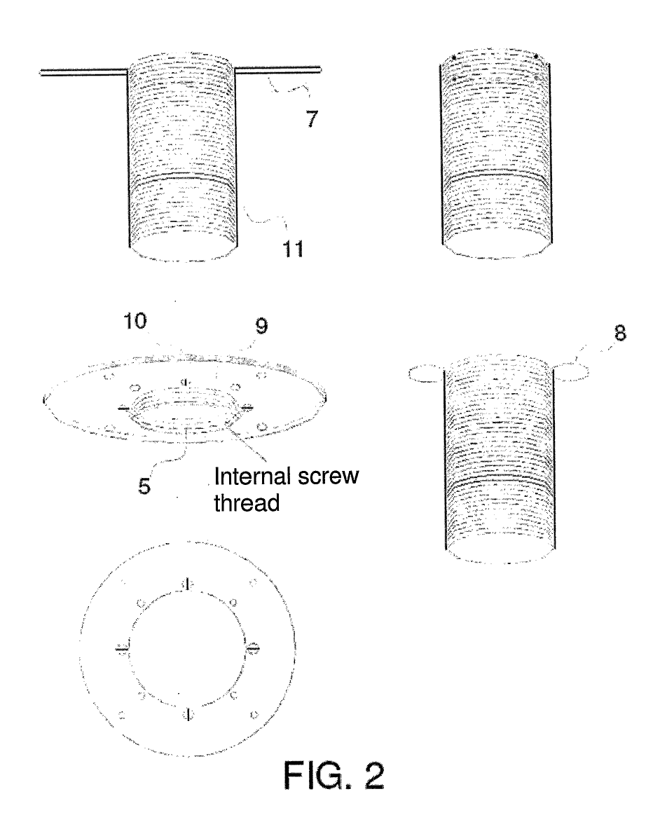

FIG. 2 represents a prosthesis with flange and a small tubular member 9. The flange 1 comprises internal and external holes and double holes 10 intercalated among internal holes, with the same objective of the prosthesis shown in FIG. 1. However, the small tubular member 9, besides being very short, it has a screw 5 inside. Other device called mandrill 11, represented by a long tubular member, containing screws in its external surface and fixed or removable rods 7, 8 in the upper end will be used to facilitate the applicability of the prosthesis with a very short intraluminal part, it is screwed in the prosthesis, perpetrating by its light. The graft or anastomotic trunk (joint of several grafts by one of the ends), pass through the prosthesis light, it is everted and covers the intraluminal part (small tubular member 9), being fixed to it by a circular point. Backwardly, the graft passes through the mandrill light where it will be screwed in the prosthesis, as increasing the intraluminal part. The graft or trunk is everted to the extent it is screwed. The points in U, opposed and with parallel legs, from the flange 1 then, they are applied in the organ wall, the incision is made in its center and the prosthesis-graft-mandrill aggregate is introduced under the light of the organ. The points are tied by fixing the flange 1 to the organ wall and performing hemostasis. The mandrill 11 is removed and the anastomosis is concluded. When the mandrill 11 is removed, the graft or trunk is tractioned to outside, supported by the small intraluminal part of the prosthesis.

Other important application of the anastomotic aggregate mentioned is for organs which walls are very thick, as the free wall of the heart. The prosthesis is fixed with graft to the heart wall after its incision. The graft or trunk is clamped in distal direction and temporarily in its end. The graft or trunk passes backwardly inside the mandrill 11 that will progress and to be screwed to the prosthesis until reaching the left or right heart cavity. The mandrill 11 is kept to avoid graft in the route of heart wall thickness in order to avoid heart compression and malfunctioning, especially during contraction. This blood flow, in left side, can be directed to, for example, to the coronaries (by doing myocardial ventricular coronary, sisto-diastolic revascularization); to carotid, upper member arteries, descending aorta, pulmonary arteries (ventricular-pulmonary central shunt), mesenteric arteries, celiac trunk etc. When put in the right ventricular cavity, the flow can be directed to pulmonary arteries, in cases of pulmonary valvar stenosis or infundibular valvar. The whole route will have a prosthesis coated with graft.

Other technical embodiment with the mentioned anastomotic aggregate is a prosthesis and mandrill 11 not coated with everted grafts, and the mandrill 11 must be long enough to remain a small part above the flange 1 of the prosthesis, to connect the graft or anastomotic trunk, coating its exterior and being fixed with an external circular point. In this case, the mandrill could be valved, allowing the blood flow outwardly only, to the extent that a left ventricular coronary revascularization, so it would be more physiological, that is, the coronary perfusion would be more diastolic and there would be no blood stolen from the coronary to the left ventricular cavity in the heart diastole, because the valve would be closes, allowing the passage only in one direction of the coronary.

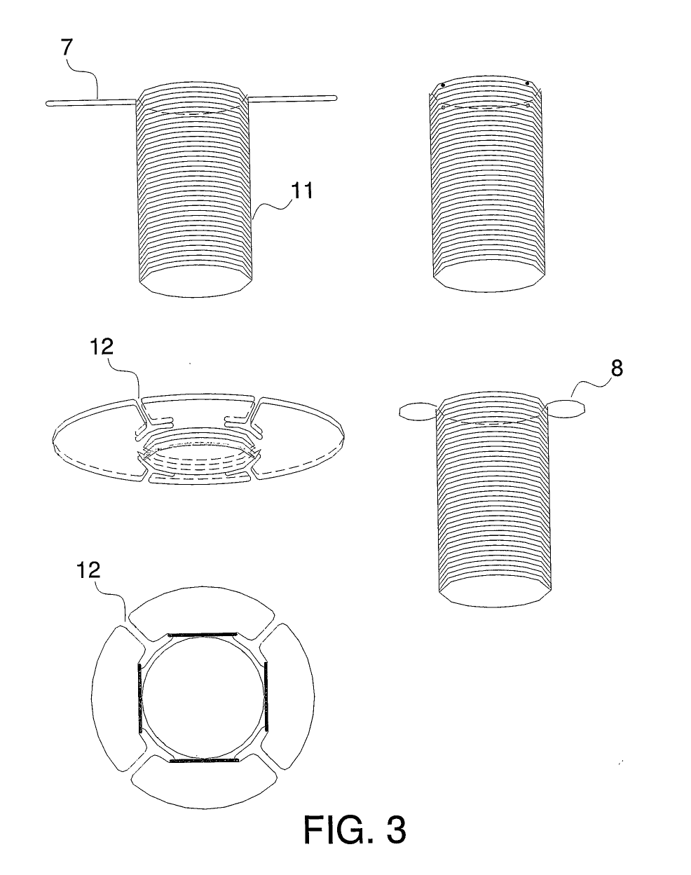

The prosthesis in FIG. 3 presents other modality of passing through openings in the flange that are not holes, neither double holes, but chamfers 12, by where the wires will pass and also impeded if interposing the light of the prosthesis, as represented. This modality facilitates the incision of the organ wall, because the handles of the wires will try only the flange 1 after the introduction of the prosthesis grafts aggregate under the light. Then the wires are tied and the mandrill is removed, finishing the anastomosis.

Therefore, this implies that it may be understood that screwed prosthesis for anastomosis and its components described above are just some of the modalities and examples of situations that would occur, the real scope of the object of invention is defined in the claims.

1. Prosthesis for anastomosis comprising: a tubular member (2), a lumen and a flange (1) extending from a side wall of the tubular member (2);
the flange (1) comprising a plurality of through openings (3) distributed on a surface thereof, wherein the flange (1) and the tubular body (2) are threaded and can be interconnected.

2. Prosthesis for anastomosis, according to claim 1 wherein the flange (1) and the tubular member (2) joined, form a prosthesis that performs anastomosis.

3. Prosthesis for anastomosis, according to claim 1, wherein an everted graft is fixed to the lower surface of the flange (1).

4. Prosthesis for anastomosis, according to claim 1, wherein the tubular member (2) has an adjustable extension by screwing and unscrewing.

5. Prosthesis for anastomosis comprising, a flange (1), a short small tubular member (9) and orthogonal to the flange (1), multiple through holes (3) to the flange (1), and an externally threaded long tubular member (11).

6. Prosthesis for anastomosis, according to claim 5, wherein the threaded long tubular member (11) is a mandrill, which is removed at the end of the anastomosis, from organs with less thick walls.

7. Prosthesis for anastomosis, according to claim 5, wherein the long tubular member (11) remains interconnected to the flange (1) in organs with thicker walls.

8. Prosthesis for anastomosis, according to claim 5, wherein the long tubular member (11) is partially threaded, limiting the upper extension of the flange (1) where the grafts are fixed.
